SEQUENCE LISTING (29 sequences)

SEQ ID NO: 1 - DNA, 30 bp, Synthetic Construct
  source 1..30: mol_type = other DNA
catccccattttgaatatttttatacgcta

SEQ ID NO: 2 - DNA, 27 bp, Synthetic Construct
  source 1..27: mol_type = other DNA
ttttatacgctaatataccggcattga

SEQ ID NO: 3 - DNA, 31 bp, Synthetic Construct
  source 1..31: mol_type = other DNA
accataaaaatcatgattgcgttattgatac

SEQ ID NO: 4 - DNA, 32 bp, Synthetic Construct
  source 1..32: mol_type = other DNA
ataccataaaaatcatgattgcgttattgata

SEQ ID NO: 5 - DNA, 30 bp, Synthetic Construct
  source 1..30: mol_type = other DNA
ataccataaaaatcatgattgcgttattga

SEQ ID NO: 6 - DNA, 33 bp, Synthetic Construct
  source 1..33: mol_type = other DNA
ttcagtatccgcatatcgtctgcagggatttat

SEQ ID NO: 7 - DNA, 24 bp, Synthetic Construct
  source 1..24: mol_type = other DNA
ctctcattgatttttcgcgggata

SEQ ID NO: 8 - DNA, 24 bp, Synthetic Construct
  source 1..24: mol_type = other DNA
tctcattgatttttcgcgggatac

SEQ ID NO: 9 - DNA, 23 bp, Synthetic Construct
  source 1..23: mol_type = other DNA
tcattgatttttcgcgggataca

SEQ ID NO: 10 - DNA, 23 bp, Synthetic Construct
  source 1..23: mol_type = other DNA
ttgatttttcgcgggatacatca

SEQ ID NO: 11 - DNA, 22 bp, Synthetic Construct
  source 1..22: mol_type = other DNA
acgacaatggatgctgatacac

SEQ ID NO: 12 - DNA, 24 bp, Synthetic Construct
  source 1..24: mol_type = other DNA
tctacgacaatggatgctgataca

SEQ ID NO: 13 - DNA, 29 bp, Synthetic Construct
  source 1..29: mol_type = other DNA
tagcatcagcatcagaatctgtaggccgt

SEQ ID NO: 14 - DNA, 24 bp, Synthetic Construct
  source 1..24: mol_type = other DNA
tcgttatgctcctgatattcggaa

SEQ ID NO: 15 - DNA, 26 bp, Synthetic Construct
  source 1..26: mol_type = other DNA
gatattcggaaatctgtggagtttca

SEQ ID NO: 16 - DNA, 24 bp, Synthetic Construct
  source 1..24: mol_type = other DNA
gtttcaggttttggtggaagtgta

SEQ ID NO: 17 - DNA, 23 bp, Synthetic Construct
  source 1..23: mol_type = other DNA
tcaggttttggtggaagtgtaac

SEQ ID NO: 18 - DNA, 19 bp, Synthetic Construct
  source 1..19: mol_type = other DNA
ggtggatgtcgaacccgaa

SEQ ID NO: 19 - DNA, 19 bp, Synthetic Construct
  source 1..19: mol_type = other DNA
accatcggtggatgtcgaa

SEQ ID NO: 20 - DNA, 21 bp, Synthetic Construct
  source 1..21: mol_type = other DNA
gattagtggcgtgacaccatc

SEQ ID NO: 21 - DNA, 37 bp, Synthetic Construct
  source 1..37: mol_type = other DNA
actctctgaaatatccttcagtatcccaccaagtagc

SEQ ID NO: 22 - DNA, 24 bp, Synthetic Construct
  source 1..24: mol_type = other DNA
gacataaacagtattgacccacga

SEQ ID NO: 23 - DNA, 26 bp, Synthetic Construct
  source 1..26: mol_type = other DNA
cataaacagtattgacccacgagtta

SEQ ID NO: 24 - DNA, 27 bp, Synthetic Construct
  source 1..27: mol_type = other DNA
cagtattgacccacgagttataacaaa

SEQ ID NO: 25 - DNA, 25 bp, Synthetic Construct
  source 1..25: mol_type = other DNA
gacccacgagttataacaaagtcaa

SEQ ID NO: 26 - DNA, 28 bp, Synthetic Construct
  source 1..28: mol_type = other DNA
ccaaaatgaattagggttgtcatatcca

SEQ ID NO: 27 - DNA, 28 bp, Synthetic Construct
  source 1..28: mol_type = other DNA
tcccaaaatgaattagggttgtcatatc

SEQ ID NO: 28 - DNA, 24 bp, Synthetic Construct
  source 1..24: mol_type = other DNA
tcccaaaatgaattagggttgtca

SEQ ID NO: 29 - DNA, 36 bp, Synthetic Construct
  source 1..36: mol_type = other DNA
acccattttcctgctccattgagtgtaaaattcatg

(19) United States
(12) Patent Application Publication
(10) Pub. No.: US 2026/0055475 A1
(43) Pub. Date: Feb. 26, 2026

(54) COMPOSITIONS AND METHODS FOR DETECTING MONKEYPOX VIRUS

(71) Applicant: Roche Molecular Systems, Inc., Pleasanton, CA (US)

(72) Inventors: Buyanzaya BuyanUrt, Alameda, CA (US); Ke Chen, Palo Alto, CA (US); Tanja Fabricius, Schwyz (CH); Jody Harris, Moraga, CA (US); Marintha Heil, Danville, CA (US); Andrew Thomas Hill, Livermore, CA (US); Jisheng Li, Pleasanton, CA (US); Thomas Meister, Sunnyvale, CA (US); Joshua Shak, Burlingame, CA (US); Jingtao Sun, San Ramon, CA (US); Manu Vanaerschot, Millbrae, CA (US); Shearing Patrick Volkir, Oro Valley, AZ (US)

(21) Appl. No.: 19/104,923

(22) PCT Filed: Aug. 22, 2023
(86) PCT No.: PCT/EP2023/072959

§ 371 Date: Feb. 19, 2025

Related U.S. Application Data
(60) Provisional application No. 63/400,734, filed on Aug. 24, 2022.
(60) Provisional application No. 63/452,618, filed on Mar. 16, 2023.

(51) Int. Cl. C12Q 1/70; C12Q 1/48; C12Q 1/6809; C12Q 1/6818; C12Q 1/686

(52) U.S. Cl. CPC C12Q 1/701; C12Q 1/48; C12Q 1/6809; C12Q 1/6818; C12Q 1/686; C12Y 207/07007; C12Q 2600/118; C12Q 2600/16

(57) ABSTRACT

Methods for the rapid detection of the presence or absence of Monkeypox Virus (MPXV) in a biological or non-biological sample are described. The methods can include performing an amplifying step, a hybridizing step, and a detecting step. Furthermore, primers, probes targeting the MPXV F3L gene and the MPXV B21R gene, along with kits are provided that are designed for the detection of MPXV.

### CROSS-REFERENCE TO RELATED APPLICATIONS

This application claims priority to and the benefit of U.S. Provisional Application No. 63/400,734, filed Aug. 24, 2022, and U.S. Provisional Application No. 63/452,618, filed Mar. 16, 20223, the contents of which are incorporated herein by reference in their entirety.

### SEQUENCE LISTING

The instant application contains a sequencing listing which has been submitted electronically in ASCII format and is hereby incorporated by reference in its entirety. Said ASCII copy, created on June Aug. 14, 2023 is named “P37722-WO PCT filing sequence listing” and is 492,822 bytes in size.

### FIELD OF THE INVENTION

The present disclosure relates to the field of molecular diagnostics, and more particularly to the detection of Monkeypox Virus (MPXV) by a polymerase chain reaction (PCR) assay.

### BACKGROUND OF THE INVENTION

Monkeypox was first described as a pox-like disease in research monkeys in 1958 and in humans in 1970, and is caused by infection with monkeypox virus (MPXV). The course of infection starts with an incubation period of 5-21 days followed by a prodromal phase in which the patient presents with general symptoms such as fever, lymphadenopathy, headache, and sore throat. After 1-3 days, skin manifestation will occur in the form of a rash and co-developing lesions. While lesions are usually self-resolving within several weeks, pitted scars or areas of lighter or darker skin may remain. Major complications can present in the form of secondary infections, bronchopneumonia, sepsis, encephalitis and infection of the cornea and loss of vision.

MPXV is a double-stranded DNA virus classified in the genus Orthopoxvirus in the family Poxviridae. Two major clades of MPXV have been described: clade I (previously referred to as the Central African clade) that is suggested to cause more severe disease and a high case fatality rate (˜10%), and clade II (previously referred to as the West African clade) suggested to cause milder disease and a lower case fatality rate (˜1%).

Historically, transmission of MPXV primarily occurred from infected reservoir mammals, such as rodents and non-human primates, to humans through contact with lesions, larger respiratory droplets or contaminated material. Cases predominantly occurred in endemic regions of West and Central Africa, with rare travel-associated cases or import-related cases elsewhere. In May 2022, however, a major change in the epidemiology of MPXV was observed. Extensive human-to-human transmission of MPXV, predominantly among men having sex with men, was described in countries that historically did not report monkeypox.

On Aug. 22, 2022, the CDC reported 41.358 confirmed cases worldwide, of which 14,115 in the United States of America. Genomic studies on the 2022 MPXV revealed a clade II origin of the virus in 2018-2019, and a higher-than-expected number of MPXV genomic mutations, suggested to be due to host-mediated microevolution (Isidro et al., Nature Medicine vol. 28, pp. 1569-1572, 2022). There are currently no approved treatments available for monkeypox, but vaccination using smallpox vaccines has shown efficacy in preventing monkeypox if administered pre-exposure or shortly after exposure, or in limiting symptoms of disease if administered several days after exposure but before onset of disease. Early detection and contact tracing are therefore key to control monkeypox.

Several PCR-based molecular tests have been developed for the detection of monkeypox virus during previous outbreaks, but are not monkeypox virus-specific as they detect also other non-variola orthopoxviruses, or show mismatches in the primer or probe compared to the MPXV responsible for the 2022 outbreak. Specific detection of monkeypox virus is important given that it is a requirement for confirmation of monkeypox as set forth by CDC case definitions. There are currently no commercial FDA-approved IVD molecular tests available for MPXV diagnosis.

### SUMMARY OF THE INVENTION

The present invention discloses a real-time polymerase chain reaction (PCR) assay to detect and optionally quantify MPXV in skin or lesion swabs, plasma or in other types of biological samples. The challenges associated with the detection of MPXV with high specificity as discussed above was solved by detecting a plurality of MPXV targets selected in silico for high inclusivity on the MPXV genome. In comparison to current lab-developed and commercial MPXV assays, this invention has advanced the field by providing an added benefit to properly detect and quantify MPXV in case one target failed detection due to rapid virus evolution.

Certain embodiments in the present disclosure relate to methods for the rapid detection of the presence or absence of MPXV in a biological or non-biological sample, for example, multiplex detection of MPXV by real-time PCR in a single test tube. Embodiments include methods of detection of MPXV comprising performing at least one cycling step, which may include an amplifying step and a hybridizing step. Furthermore, embodiments include primers, probes, and kits that are designed for the detection of MPXV in a single reaction vessel (e.g. a tube or a well). The detection methods are designed to target two, three or four target regions with optimal inclusivity for all MPXV genomes and exclusivity to other orthopoxviruses, which allows one to detect MPXV in a single test.

In one aspect, a method for detecting at least two target nucleic acids of MPXV in a sample is provided, including (a) providing a sample; (b) performing an amplification step comprising contacting the sample with at least two sets of primers to produce amplification products, if the at least two target nucleic acids of MPXV are present in the sample; (c) performing a hybridization step, comprising contacting the amplification products, if the at least two target nucleic acids of MPXV is present in the sample, with at least two probes; and (d) performing a detection step, comprising detecting the presence or absence of the amplification products, wherein the presence of one of the amplification products is indicative of the presence of MPXV in the sample, and wherein the absence of the amplification product is indicative of the absence of MPXV in the sample. In a related embodiment, the at least two target nucleic acids of MPXV are selected from the group consisting of a gene encoding a serine protease inhibitor-like protein (C2L gene), a gene encoding a putative double-stranded RNA binding protein (F3L gene), an intergenic non-coding sequence between the A25R and A26L genes (INCS), and a gene encoding an immunogenic membrane-associated glycoprotein (B21R gene). In another related embodiment, the at least two target nucleic acids of MPXV are the F3L gene and the B21R gene. In one embodiment, a first set of primers produces one or more amplification products of a first target nucleic acid that are detected by a first probe or a first set of probes and a second set of primers produces one or more amplification products of a second target nucleic acid that are detected by a second probe or a second set of probes. In one embodiment, the first target nucleic acid is the MPXV F3L gene and the second target nucleic acid is the MPXV B21R gene. In some embodiments, the at least two sets of oligonucleotide primers and the at least two oligonucleotide probes comprise (i) a first set of oligonucleotide primers for amplification of the F3L gene target nucleic acid comprising a forward primer comprising or consisting of a nucleic acid sequence selected from the group consisting of SEQ ID NOs: 7-10, or any combination of forward primers each comprising or consisting of a nucleic acid sequence selected from the group consisting of SEQ ID NOs: 7-10; and a reverse primer comprising or consisting of a nucleic acid sequence selected from the group consisting of SEQ ID NOs: 11-12, or a combination thereof; and a first probe or a first set of probes for detection of an amplification product of the F3L gene target nucleic acid comprising or consisting of a nucleic acid sequence of SEQ ID NO: 13, or a complement thereof; and (ii) a second set of oligonucleotide primers for amplification of the B21R gene target nucleic acid comprising a forward primer comprising or consisting of a nucleic acid sequence selected from the group consisting of SEQ ID NOs: 22-25, or any combination of forward primers each comprising or consisting of a nucleic acid sequence selected from the group consisting of SEQ ID NOs: 22-25; and a reverse primer comprising or consisting of a nucleic acid sequence selected from the group consisting of SEQ ID NOs: 26-28, or any combination of reverse primers each comprising or consisting of a nucleic acid sequence selected from the group consisting of SEQ ID NOs: 22-25; and a second probe or a second set of probes for detection of an amplification product of the B21R gene target nucleic acid comprising or consisting of a nucleic acid sequence of SEQ ID NO: 29, or a complement thereof. In another related embodiment, the first set of oligonucleotide primers for amplification of the F3L gene target nucleic acid comprises a forward primer comprising or consisting of a nucleic acid sequence of SEQ ID NO: 7 and a reverse primer comprising or consisting of a nucleic acid sequence of SEQ ID NO: 11, and the second set of oligonucleotide primers of amplification of the B21R gene target nucleic acid comprises a forward primer comprising or consisting of a nucleic acid sequence of SEQ ID NO: 25 and a reverse primer comprising or consisting of a nucleic acid sequence of SEQ ID NO: 26. In some embodiments, the hybridization step comprises contacting the amplification products with the at least two oligonucleotide probes that are each labeled with a donor fluorescent moiety and a corresponding acceptor moiety; and the detection step comprises detecting the presence or absence of fluorescence resonance energy transfer (FRET) between the donor fluorescent moiety and the acceptor moiety of the probes, wherein the presence or absence of fluorescence is indicative of the presence or absence of MPXV in the sample. In some embodiments, the detectable probes are each labeled with a same donor fluorescent moiety. In other embodiments, the detectable probes are each labeled with a different donor fluorescent moiety. In some embodiments, the amplification step employs a polymerase enzyme having 5′ to 3′ nuclease activity. In some embodiments, the sample is a biological sample. In a related embodiment, the biological sample is skin swab, lesion swab or plasma.

In another aspect, a method for detecting MPXV in a sample is provided, including (a) performing an amplification step comprising contacting the sample with a one or more forward oligonucleotide primers and one or more reverse oligonucleotide primers specifically hybridizing to the MPXV F3L gene to produce amplification products of the F3L gene if MPXV is present in the sample; and one or more forward oligonucleotide primers and one or more reverse oligonucleotide primers specifically hybridizing to the MPXV B21R gene to produce amplification products of the B21R gene if MPXV is present in the sample; (b) performing a hybridization step comprising contacting the F3L gene amplification products with one or more detectable oligonucleotide probes specifically hybridizing to the F3L gene amplification products and contacting the B21R gene amplification products with one or more detectable oligonucleotide probes specifically hybridizing to the B21R gene amplification products; and (c) detecting the presence or absence of the F3L gene amplification products and/or the B21R gene amplification products, wherein the presence of either the F3L gene amplification products or the B21R amplification products or both amplification products is indicative of the presence of MPXV in the sample and wherein the absence of both the F3L gene amplification products and the B21R amplification products is indicative of the absence of MPXV in the sample; wherein one of the one or more F3L gene forward oligonucleotide primers comprises or consists of a nucleic acid sequence selected from SEQ ID NOs: 7-10, or the one or more F3L gene forward oligonucleotide primers comprise any combination of forward primers each comprising or consisting of a nucleic acid sequence selected from the group consisting of SEQ ID NOs: 7-10; and one of the one or more F3L gene reverse oligonucleotide primers comprises or consists of a nucleic acid sequence selected from SEQ ID NOs: 11-12, or the one or more F3L gene reverse oligonucleotide primers comprise two reverse primers comprising or consisting of a nucleic acid sequence of SEQ ID NOs: 11 and 12; and one of the one or more detectable F3L gene oligonucleotide probes comprises or consists of a nucleic acid sequence of SEQ ID NO: 13, or a complement thereof; and wherein one of the one or more B21R gene forward oligonucleotide primers comprises or consists of a nucleic acid sequence selected from SEQ ID NOs: 22-25, or the one or more B21R gene forward oligonucleotide primers comprise any combination of forward primers each comprising or consisting of a nucleic acid sequence selected from the group consisting of SEQ ID NOs: 22-25; and one of the one or more B21R gene reverse oligonucleotide primers comprises or consists of a nucleic acid sequence selected from SEQ ID NOs: 26-28, or the one or more B21R gene reverse oligonucleotide primers comprise any combination of reverse primers each comprising or consisting of a nucleic acid sequence selected from the group consisting of SEQ ID NOs: 26-28; and one of the one or more detectable B21R gene oligonucleotide probes comprises or consists of a nucleic acid sequence of SEQ ID NO: 29, or a complement thereof. In some embodiments, one of the one or more F3L gene forward oligonucleotide primers comprises or consists of the nucleic acid sequence of SEQ ID NO: 7, and one of the one or more F3L gene reverse oligonucleotide primers comprises or consists of the nucleic acid sequence of SEQ ID NO: 11. In some embodiments, the one or more B21R gene forward oligonucleotide primers comprises or consists of the nucleic acid sequence of SEQ ID NO: 25, and one of the one or more B21R gene reverse oligonucleotide primers comprises or consists of the nucleic acid sequence of SEQ ID NO: 26. In some embodiments, the hybridization step comprises contacting the amplification products with the at least two oligonucleotide probes that are each labeled with a donor fluorescent moiety and a corresponding acceptor moiety; and the detection step comprises detecting the presence or absence of fluorescence resonance energy transfer (FRET) between the donor fluorescent moiety and the acceptor moiety of the probes, wherein the presence or absence of fluorescence is indicative of the presence or absence of MPXV in the sample. In some embodiments, the detectable probes are each labeled with a same donor fluorescent moiety. In other embodiments, the detectable probes are each labeled with a different donor fluorescent moiety. In some embodiments, the amplification step employs a polymerase enzyme having 5′ to 3′ nuclease activity. In a related embodiment, the sample is a biological sample. In a related embodiment, the biological sample is skin swab, lesion swab or plasma. In one embodiment, the sample is a biological sample. In another embodiment, the biological sample is blood, plasma or serum.

In some embodiments of the methods disclosed herein, amplification can employ a polymerase enzyme having 5′ to 3′ nuclease activity. Thus, the label moiety and quencher moiety, which are the first and second fluorescent moieties, may be within no more than 8 nucleotides of each other along the length of the probe. In another aspect, the F3L gene and/or the B21R gene probes include a nucleic acid sequence that permits secondary structure formation. Such secondary structure formation generally results in spatial proximity between the first and second fluorescent moiety. According to this method, the second fluorescent moiety on the probe can be a quencher. In some embodiments, the F3L and B21R gene probes may be labeled with a donor fluorescent dye that acts as a reporter. The probe may also have a second dye that acts as a quencher. The donor fluorescent dye is measured at a defined wavelength, thus permitting detection and discrimination of the amplified MPXV F3L and B21R gene targets. In one embodiment, the probes are each labeled with a same donor fluorescent dye. In another embodiment, the probes are each labeled with a different donor fluorescent dye. The fluorescent signal of the intact probes is suppressed by the quencher dye. During the PCR amplification step, hybridization of the probes to the specific single-stranded DNA template results in cleavage by the 5′ to 3′ nuclease activity of the DNA polymerase resulting in separation of the reporter and quencher dyes and the generation of a fluorescent signal. With each PCR cycle, increasing amounts of cleaved probes are generated and the cumulative signal of the reporter dye is concomitantly increased. Optionally, one or more additional probes (e.g., such as an internal reference control or other targeted probe (e.g., other viral nucleic acids) may also be labeled with a reporter fluorescent dye, unique and distinct from the fluorescent dye label associated with the F3L and B21R gene probes. In such case, because the specific reporter dyes are measured at defined wavelengths, simultaneous detection and discrimination of the amplified targets and the one or more additional probes is possible.

The present disclosure provides methods of detecting the presence or absence of MPXV or MPXV nucleic acid, in a biological sample from an individual. These methods can be employed to detect the presence or absence of MPXV or MPXV nucleic acid in biological samples such as skin swabs, lesion swabs, serum, plasma, or other biological materials believed to have MPXV present, for use in diagnostic testing. Additionally, the same test may be used by someone experienced in the art to assess other sample types to detect MPXV or MPXV nucleic acid. Such methods optionally include performing a reverse transcription step and at least one cycling step, which includes an amplifying step and either a detectable probe binding step or a dye-binding step. Typically, the amplifying step includes contacting the sample with a plurality of pairs of oligonucleotide primers to produce one or more amplification products if a nucleic acid molecule is present in the sample, the probe binding step includes contacting the amplification product with one or more detectable probes specific for the amplification product, and the dye-binding step includes contacting the amplification product with a double-stranded DNA binding dye. Such methods also include detecting the presence or absence of binding of the double-stranded DNA binding dye into the amplification product, wherein the presence of binding is indicative of the presence of MPXV or MPXV nucleic acid in the sample, and wherein the absence of binding is indicative of the absence of MPXV or MPXV nucleic acid in the sample. A representative double-stranded DNA binding dye is ethidium bromide. Other nucleic acid-binding dyes include DAPI, Hoechst dyes, PicoGreen®, RiboGreen®, OliGreen®, and cyanine dyes such as YO-YO® and SYBR® Green. In addition, such methods also can include determining the melting temperature between the amplification product and the double-stranded DNA binding dye, wherein the melting temperature confirms the presence or absence of MPXV or MPXV nucleic acid.

Also disclosed is that the at least two sets of primers and the at least two probes comprise: (i) a first set of primers comprising a forward primer comprising a nucleic acid sequence of SEQ ID NOs: 1-2 or any combination of SEQ ID NOs: 1-2 thereof; and a reverse primer comprising a nucleic acid sequence of SEQ ID NOs: 3-5, or a combination thereof; and a first probe or a first set of probes comprising a nucleic acid sequence of SEQ ID NO: 6, or a complement thereof; and (ii) a second set of primers comprising a forward primer comprising a nucleic acid sequence of SEQ ID NOs: 7-10, or a combinations thereof; and a reverse primer comprising a nucleic acid sequence of SEQ ID NOs: 11-12, or a combination thereof; and a second probe or a second set of probes comprising a nucleic acid sequence of SEQ ID NO: 13 or a complement thereof.

In a further aspect, a kit for detecting a first target nucleic acid within the F3L gene of MPXV and a second target nucleic acid within the B21R gene of MPXV in a sample is provided, the kit comprising amplification reagents comprising (a) a DNA polymerase having 5′ to 3′ nuclease activity; (b) nucleoside triphosphates; (c) a first set of oligonucleotide primers and a first oligonucleotide probe or first set of oligonucleotide probes for amplifying and detecting the F3L gene target nucleic acid of MPXV, wherein the first set of oligonucleotide primers for amplification of the F3L gene target nucleic acid comprises a forward primer comprising or consisting of a nucleic acid sequence selected from the group consisting of SEQ ID NOs: 7-10, or any combination of forward primers each comprising or consisting of a nucleic acid sequence selected from the group consisting of SEQ ID NOs: 7-10; and a reverse primer comprising or consisting of a nucleic acid sequence selected from the group consisting of SEQ ID NOs: 11-12, or a combination thereof; and wherein the first probe or first set of probes for detection of an amplification product of the F3L gene target nucleic acid comprises or consists of a nucleic acid sequence of SEQ ID NO: 13, or a complement thereof; and (d) a second set of oligonucleotide primers and a second oligonucleotide probe or second set of oligonucleotide probes for amplifying and detecting the B21R gene target nucleic acid of MPXV, wherein the second set of oligonucleotide primers for amplification of the B21R gene target nucleic acid comprises a forward primer comprising or consisting of a nucleic acid sequence selected from the group consisting of SEQ ID NOs: 22-25, or any combination of forward primers each comprising or consisting of a nucleic acid sequence selected from the group consisting of SEQ ID NOs: 22-25; and a reverse primer comprising or consisting of a nucleic acid sequence selected from the group consisting of SEQ ID NOs: 26-28, or any combination of reverse primers each comprising or consisting of a nucleic acid sequence selected from the group consisting of SEQ ID NOs: 22-25; and wherein the second probe or second set of probes for detection of an amplification product of the B21R gene target nucleic acid comprises or consists of a nucleic acid sequence of SEQ ID NO: 29, or a complement thereof. In one embodiment, the kit can include probes already labeled with donor and corresponding acceptor fluorescent moieties, or can include fluorophoric moieties for labeling the probes. The kit can also include buffers necessary for the function of the nucleic acid polymerase. The kit can also include a package insert and instructions for using the primers, probes, and fluorophoric moieties to detect the presence or absence of MPXV in a sample. In some embodiments, the first and second oligonucleotide probes or the first set and second set of oligonucleotide probes are labeled with a donor fluorescent moiety and a corresponding acceptor moiety. In certain embodiments, the first and second oligonucleotide probes are each labeled with a same donor fluorescent moiety. In other embodiments, the first and second oligonucleotide probes are each labeled with a different donor fluorescent moiety.

In another aspect, an oligonucleotide is provided comprising or consisting of a sequence of nucleotides selected from SEQ ID NOs: 1-29, or a complement thereof, which oligonucleotide has 100 or fewer nucleotides. In some embodiments, an oligonucleotide is provided comprising or consisting of a sequence of nucleotides selected from SEQ ID NOs: 7-13 and 22-29, or a complement thereof, which oligonucleotide has 100 or fewer nucleotides. The present disclosure further provides an oligonucleotide that includes a nucleic acid having at least 70% sequence identity (e.g., at least 75%, 80%, 85%, 90% or 95%, etc.) to one of SEQ ID NOs: 1-29, or a complement thereof, which oligonucleotide has 100 or fewer nucleotides. Generally, these oligonucleotides may be primer nucleic acids, probe nucleic acids, or the like in these embodiments. In certain of these embodiments, the oligonucleotides have 40 or fewer nucleotides (e.g., 35 or fewer nucleotides, 30 or fewer nucleotides, 25 or fewer nucleotides, 20 or fewer nucleotides, 15 or fewer nucleotides, etc.) In some embodiments, the oligonucleotides comprise at least one modified nucleotide, e.g., to alter nucleic acid hybridization stability relative to unmodified nucleotides. Optionally, the oligonucleotides comprise at least one label moiety and optionally at least one quencher moiety. In some embodiments, the at least one label moiety and the at least one quencher moiety are fluorescent moieties. In some embodiments, the oligonucleotides include at least one conservatively modified variation. “Conservatively modified variations” or, simply, “conservative variations” of a particular nucleic acid sequence refers to those nucleic acids, which encode identical or essentially identical amino acid sequences, or, where the nucleic acid does not encode an amino acid sequence, to essentially identical sequences. One of skill in the art will recognize that individual substitutions, deletions or additions which alter, add or delete a single nucleotide or a small percentage of nucleotides (typically less than 5%, more typically less than 4%, 2% or 1%) in an encoded sequence are “conservatively modified variations” where the alterations result in the deletion of an amino acid, addition of an amino acid, or substitution of an amino acid with a chemically similar amino acid. Unless otherwise defined, all technical and scientific terms used herein have the same meaning as commonly understood by one of ordinary skill in the art to which this invention belongs. Although methods and materials similar or equivalent to those described herein can be used in the practice or testing of the present subject matter, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### BRIEF DESCRIPTION OF THE DRAWINGS

FIG. 1 is a schematic illustration of the relative locations of the primers and probes used for amplification and detection of the MPXV gene targets relative to GenBank Accession No. NC 063383, a full genomic sequence of a clade II strain published by CDC. The element F3 denotes to the C2L gene region, the element F5 denotes the F3L gene region, the element F14 denotes the intragenic non-coding sequence, and the element F19 denotes the B21R gene region.

FIG. 2 is a table illustrating the results of a linearity study for one example of a prototype MPXV PCR assay performed using the set of primers and probes according to the present disclosure.

FIG. 3 is table showing the results of a limit of detection (LoD) study for one example of a prototype MPXV PCR assay performed using the set of primers and probes according to the present disclosure.

### DETAILED DESCRIPTION OF THE INVENTION

### I. Definitions

As used herein, the term “amplifying” refers to the process of synthesizing nucleic acid molecules that are complementary to one or both strands of a template nucleic acid molecule (e.g., MPXV F3L gene or MPXV B21R gene). Amplifying a nucleic acid molecule typically includes denaturing the template nucleic acid, annealing primers to the template nucleic acid at a temperature that is below the melting temperatures of the primers, and enzymatically elongating from the primers to generate an amplification product. Amplification typically requires the presence of deoxyribonucleoside triphosphates, a DNA polymerase enzyme (e.g., Platinum® Taq) and an appropriate buffer and/or co-factors for optimal activity of the polymerase enzyme (e.g., MgCl₂ and/or KCl).

The term “primer” is used herein as known to those skilled in the art and refers to oligomeric compounds, primarily to oligonucleotides but also to modified oligonucleotides that are able to “prime” DNA synthesis by a template-dependent DNA polymerase, i.e., the 3′-end of the, e.g., oligonucleotide provides a free 3′—OH group whereto further “nucleotides” may be attached by a template-dependent DNA polymerase establishing 3′ to 5′ phosphodiester linkage whereby deoxynucleoside triphosphates are used and whereby pyrophosphate is released. Therefore, there is-except possibly for the intended function-no fundamental difference between a “primer”, an “oligonucleotide”, or a “probe”.

The term “probe” refers to synthetically or biologically produced nucleic acids (DNA or RNA), which by design or selection, contain specific nucleotide sequences that allow them to hybridize under defined predetermined stringencies specifically (i.e., preferentially) to “target nucleic acids”. A “probe” can be referred to as a “detection probe” meaning that it detects the target nucleic acid.

The term “hybridizing” refers to the annealing of one or more probes to an amplification product.

Hybridization conditions typically include a temperature that is below the melting temperature of the probes but that avoids non-specific hybridization of the probes.

The term “5′ to 3′ nuclease activity” refers to an activity of a nucleic acid polymerase, typically associated with the nucleic acid strand synthesis, whereby nucleotides are removed from the 5′ end of nucleic acid strand.

The term “thermostable polymerase” refers to a polymerase enzyme that is heat stable, i.e., the enzyme catalyzes the formation of primer extension products complementary to a template and does not irreversibly denature when subjected to the elevated temperatures for the time necessary to effect denaturation of double-stranded template nucleic acids. Generally, the synthesis is initiated at the 3′ end of each primer and proceeds in the 5′ to 3′ direction along the template strand. Thermostable polymerases have been isolated from Thermus flavus, T. ruber, T. thermophilus, T. aquaticus, T. lacteus, T. rubens, Bacillus stearothermophilus, and Methanothermus fervidus. Nonetheless, polymerases that are not thermostable also can be employed in PCR assays provided the enzyme is replenished.

The term “complement thereof” refers to nucleic acid that is both the same length as, and exactly complementary to, a given nucleic acid.

The term “extension” or “elongation” when used with respect to nucleic acids refers to when additional nucleotides (or other analogous molecules) are incorporated into the nucleic acids. For example, a nucleic acid is optionally extended by a nucleotide incorporating biocatalyst, such as a polymerase that typically adds nucleotides at the 3′ terminal end of a nucleic acid.

The terms “identical” or percent “identity” in the context of two or more nucleic acid sequences, refer to two or more sequences or subsequences that are the same or have a specified percentage of nucleotides that are the same, when compared and aligned for maximum correspondence, e.g., as measured using one of the sequence comparison algorithms available to persons of skill or by visual inspection. Exemplary algorithms that are suitable for determining percent sequence identity and sequence similarity are the BLAST programs, which are described in, e.g., Altschul et al. (1990) “Basic local alignment search tool” J. Mol. Biol. 215:403-410, Gish et al. (1993) “Identification of protein coding regions by database similarity search” Nature Genet. 3:266-272, Madden et al. (1996) “Applications of network BLAST server” Meth. Enzymol. 266:131-141, Altschul et al. (1997) “Gapped BLAST and PSI-BLAST: a new generation of protein database search programs” Nucleic Acids Res. 25:3389-3402, and Zhang et al. (1997) “PowerBLAST: A new network BLAST application for interactive or automated sequence analysis and annotation” Genome Res. 7:649-656, which are each incorporated herein by reference.

A “modified nucleotide” in the context of an oligonucleotide refers to an alteration in which at least one nucleotide of the oligonucleotide sequence is replaced by a different nucleotide that provides a desired property to the oligonucleotide. Exemplary modified nucleotides that can be substituted in the oligonucleotides described herein include, e.g., a C5-methyl-dC, a C5-ethyl-dC, a C5-methyl-dU, a C5-ethyl-dU, a 2,6-diaminopurine, a C5-propynyl-dC, a C5-propynyl-dU, a C7-propynyl-dA, a C7-propynyl-dG, a C5-propargylamino-dC, a C5-propargylamino-dU, a C7-propargylamino-dA, a C7-propargylamino-dG, a 7-deaza-2-deoxyxanthosine, a pyrazolopyrimidine analog, a pseudo-dU, a nitro pyrrole, a nitro indole, 2′-0-methyl Ribo-U, 2′-0-methyl Ribo-C, an N4-ethyl-dC, an N6-methyl-dA, t-butyl-benzyl-dA, t-butyl-benzyl-dC and the like. Many other modified nucleotides that can be substituted in the oligonucleotides are referred to herein or are otherwise known in the art. In certain embodiments, modified nucleotide substitutions modify melting temperatures (Tm) of the oligonucleotides relative to the melting temperatures of corresponding unmodified oligonucleotides. To further illustrate, certain modified nucleotide substitutions can reduce non-specific nucleic acid amplification (e.g., minimize primer dimer formation or the like), increase the yield of an intended target amplicon, and/or the like in some embodiments. Examples of these types of nucleic acid modifications are described in, e.g., U.S. Pat. No. 6,001,611, which is incorporated herein by reference. Examples of these types of nucleic acid modifications are described in, e.g., U.S. Pat. No. 6,001,611, which is incorporated herein by reference.

As used herein, the term “host cell” is meant to include prokaryotes and eukaryotes such as yeast, plant and animal cells.

### II. Detailed Description of Certain Embodiments

Diagnosis of MPXV infection by nucleic acid amplification provides a method for rapidly and accurately detecting the viral infection. A real-time polymerase chain reaction (PCR) assay for detecting MPXV in a sample is described herein. Primers and probes for detecting MPXV are provided, as are articles of manufacture or kits containing such primers and probes. The increased sensitivity of real-time PCR for detection of MPXV compared to other methods, as well as the improved features of real-time PCR including sample containment and real-time detection of the amplified product, make feasible the implementation of this technology for routine diagnosis of MPXV infections in the clinical laboratory.

Genetic analysis of the 2022 MPXV genomes and other MPXV genomes identified four target regions with optimal inclusivity for all MPXV genomes and exclusivity to other orthopoxviruses. The first target region is located in a gene encoding a serine protease inhibitor-like protein (C2L). The second target region is located in a gene encoding a truncated version of the vaccinia virus E3 protein, a double-stranded RNA binding protein (F3L) known to be important for blocking activation of the host's cellular innate immune system (Arndt et al., 2015). The third target region is located in an intergenic non-coding sequence (INCS) between the A25R and A26L genes. The fourth target region is located in a gene encoding an immunogenic membrane-associated glycoprotein (B21R).

The disclosed methods may include performing at least one cycling step that includes amplifying one or more portions of MPXV F3L gene nucleic acid target and MPXV B21R gene nucleic acid target from a sample using one or more pairs of F3L gene primers and/or one or more pairs of B21R gene primers. “F3L gene primers” or “B21R primers” as used herein refer to oligonucleotide primers that specifically anneal to nucleic acid sequence in the F3L gene and the B21R gene, respectively, and initiate DNA synthesis therefrom under appropriate conditions. Each of the discussed F3L gene or B21R gene primers anneals to a target within or adjacent to the respective target nucleic acid molecule such that at least a portion of each amplification product contains nucleic acid sequence corresponding to the target. The one or more of the F3L gene amplification products and/or the B21R gene amplification products are produced provided that one or more of the F3L gene nucleic acid and/or the B21R gene nucleic acid is present in the sample, thus the presence of these one or more of amplification products is indicative of the presence of MPXV in the sample. The amplification product should contain the nucleic acid sequences that are complementary to one or more detectable probes for the F3L gene or for the B21R gene. Each cycling step includes an amplification step, a hybridization step, and a detection step, in which the sample is contacted with the one or more detectable probes for the F3L gene or for the B21R gene for detection of the presence or absence of MPXV in the sample.

The present disclosure provides methods to detect Monkeypox virus (MPXV) by amplifying, for example, a portion of the MPXV F3L gene nucleic acid sequence and/or the MPXV B21R gene nucleic acid sequence. Nucleic acid sequences of various clades of MPXV are available in GenBank (e.g. Accession No. NC_063383). Specifically, primers and probes to amplify and detect the F3L gene and the B21R gene nucleic acid molecule targets are provided by the embodiments in the present disclosure.

For detection of MPXV, primers and probes to amplify the target MPXV genes and intergenic sequence are provided. MPXV nucleic acids other than those exemplified herein can also be used to detect MPXV in a sample. For example, functional variants can be evaluated for specificity and/or sensitivity by those of skill in the art using routine methods. Representative functional variants can include, e.g., one or more deletions, insertions, and/or substitutions in the MPXV nucleic acids disclosed herein.

More specifically, embodiments of the oligonucleotides each include a nucleic acid with a sequence selected from SEQ ID NOs: 1-29, a substantially identical variant thereof in which the variant has at least, e.g., 80%, 90%, or 95% sequence identity to one of SEQ ID NOs: 1-29, or a complement of SEQ ID NOs: 1-29, and the variant.

TABLE I
C2L gene Primers and Probes

| SEQ ID NO | NAME | DESCRIPTION | SEQUENCE |
|---|---|---|---|
| 1 | MKPX3-F1 | Forward primer | CATCCCCATTTTGAATATTTTTATA |
| | | | CGCTA |
| 2 | MKPX3-F2 | Forward primer | TTTTATACGCTAATATACCGGCATT |
| | | | GA |
| 3 | MKPX3-R1 | Reverse primer | ACCATAAAAATCATGATTGCGTTA |
| | | | TTGATAC |
| 4 | MKPX3-R2 | Reverse primer | ATACCATAAAAATCATGATTGCGT |
| | | | TATTGATA |
| 5 | MKPX3-R3 | Reverse primer | ATACCATAAAAATCATGATTGCGT |
| | | | TATTGA |
| 6 | MKPX3- | Probe | TTCAGTATCCGCATATCGTCTGCAG |
| | Probe | | GGATTTAT |

TABLE II
F3L gene Primers and Probes

| SEQ ID NO | NAME | DESCRIPTION | SEQUENCE |
|---|---|---|---|
| 7 | MKPX5-F1 | Forward primer | CTCTCATTGATTTTTCGCGGGAT |
| | | | A |
| 8 | MKPX5-F2 | Forward primer | TCTCATTGATTTTTCGCGGGATA |
| | | | C |
| 9 | MKPX5-F3 | Forward primer | TCATTGATTTTTCGCGGGATAC |
| | | | A |
| 10 | MKPX5-F4 | Forward primer | TTGATTTTTCGCGGGATACATC |
| | | | A |
| 11 | MKPX5-R1 | Reverse primer | ACGACAATGGATGCTGATACAC |
| 12 | MKPX5-R2 | Reverse primer | TCTACGACAATGGATGCTGATA |
| | | | CA |
| 13 | MKPX5-Probe | Probe | TAGCATCAGCATCAGAATCTGT |
| | | | AGGCCGT |

TABLE III
INCS Primers and Probes

| SEQ ID NO | NAME | DESCRIPTION | SEQUENCE |
|---|---|---|---|
| 14 | MKPX14-F1 | Forward primer | TCGTTATGCTCCTGATATTCGGA |
| | | | A |
| 15 | MKPX14-F2 | Forward primer | GATATTCGGAAATCTGTGGAGTT |
| | | | TCA |
| 16 | MKPX14-F3 | Forward primer | GTTTCAGGTTTTGGTGGAAGTGT |
| | | | A |
| 17 | MKPX14-F4 | Forward primer | TCAGGTTTTGGTGGAAGTGTAAC |
| 18 | MKPX14-R1 | Reverse primer | GGTGGATGTCGAACCCGAA |
| 19 | MKPX14-R2 | Reverse primer | ACCATCGGTGGATGTCGAA |
| 20 | MKPX14-R3 | Reverse primer | GATTAGTGGCGTGACACCATC |
| 21 | MKPX14- | Probe | ACTCTCTGAAATATCCTTCAGTA |
| | Probe | | TCCCACCAAGTAGC |

TABLE IV
B21R gene Primers and Probes

| SEQ ID NO | NAME | DESCRIPTION | SEQUENCE |
|---|---|---|---|
| 22 | MKPX19-F1 | Forward primer | GACATAAACAGTATTGACCCA |
| | | | CGA |
| 23 | MKPX19-F2 | Forward primer | CATAAACAGTATTGACCCACG |
| | | | AGTTA |
| 24 | MKPX19-F3 | Forward primer | CAGTATTGACCCACGAGTTAT |
| | | | AACAAA |
| 25 | MKPX5-F4 | Forward primer | GACCCACGAGTTATAACAAA |
| | | | GTCAA |
| 26 | MKPX19-R1 | Reverse primer | CCAAAATGAATTAGGGTTGTC |
| | | | ATATCCA |
| 27 | MKPX19-R2 | Reverse primer | TCCCAAAATGAATTAGGGTTG |
| | | | TCATATC |
| 28 | MKPX19-R3 | Reverse primer | TCCCAAAATGAATTAGGGTTG |
| | | | TCA |
| 29 | MKPX19- | Probe | ACCCATTTTCCTGCTCCATTG |
| | Probe | | AGTGTAAAATTCATG |

In one embodiment, the above-described sets of MPXV gene primers and probes are used in order to provide for detection of MPXV in a biological sample suspected of containing MPXV. The sets of primers and probes may comprise or consist the primers and probes specific for the C2L gene, the F3L gene, INCS or for the B21R gene nucleic acid sequences, comprising or consisting of the nucleic acid sequences of SEQ ID NOs: 1-29. In another embodiment, the 10 primers and probes for the MPXV gene targets comprise or consist of a functionally active variant of any of the primers and probes of SEQ ID NOs: 1-29.

A functionally active variant of any of the primers and/or probes of SEQ ID NOs: 1-29 may be identified by using the primers and/or probes in the disclosed methods. A functionally active variant of a primer and/or probe of any of the SEQ ID NOs: 1-29 pertains to a primer and/or probe which provides a similar or higher specificity and sensitivity in the described method or kit as compared to the respective sequence of SEQ ID NOs: 1-29.

The variant may, e.g., vary from the sequence of SEQ ID NOs: 1-29 by one or more nucleotide additions, deletions or substitutions such as one or more nucleotide additions, deletions or substitutions at the 5′ end and/or the 3′ end of the respective sequence of SEQ ID NOs: 1-29. As detailed above, a primer (and/or probe) may be chemically modified, i.e., a primer and/or probe may comprise a modified nucleotide or a non-nucleotide compound. A probe (or a primer) is then a modified oligonucleotide. “Modified nucleotides” (or “nucleotide analogs”) differ from a natural “nucleotide” by some modification but still consist of a base or base-like compound, a pentofuranosyl sugar or a pentofuranosyl sugar-like compound, a phosphate portion or phosphate-like portion, or combinations thereof. For example, a “label” may be attached to the base portion of a “nucleotide” whereby a “modified nucleotide” is obtained. A natural base in a “nucleotide” may also be replaced by, e.g., a 7-desazapurine whereby a “modified nucleotide” is obtained as well. The terms “modified nucleotide” or “nucleotide analog” are used interchangeably in the present application. A “modified nucleoside” (or “nucleoside analog”) differs from a natural nucleoside by some modification in the manner as outlined above for a “modified nucleotide” (or a “nucleotide analog”).

Oligonucleotides including modified oligonucleotides and oligonucleotide analogs that amplify a nucleic acid molecule from the MPXV gene nucleic acid sequences can be designed using, for example, a computer program such as OLIGO (Molecular Biology Insights Inc., Cascade, Colo.). Important features when designing oligonucleotides to be used as amplification primers include, but are not limited to, an appropriate size amplification product to facilitate detection (e.g., by electrophoresis), similar melting temperatures for the members of a pair of primers, and the length of each primer (i.e., the primers need to be long enough to anneal with sequence-specificity and to initiate synthesis but not so long that fidelity is reduced during oligonucleotide synthesis). Typically, oligonucleotide primers are 8 to 50 nucleotides in length (e.g., 8, 10, 12, 14, 16, 18, 20, 22, 24, 26, 28, 30, 32, 34, 36, 38, 40, 42, 44, 46, 48, or 50 nucleotides in length).

In addition to a set of primers, the methods may use one or more probes in order to detect the presence or absence of MPXV. According to the present disclosure, a “probe” may contain specific nucleotide sequences that allow them to hybridize under defined predetermined stringencies specifically to a MPXV C2L gene (target) nucleic acid, a MPXV F3L gene (target) nucleic acid, a MPXV intergenic non-coding sequence (INCS) between the A25R and A26Lgenes (target) nucleic acid and/or to a MPXV B21R gene (target) nucleic acid.

In some embodiments, the described MPXV gene probes can be labeled with at least one fluorescent label. In one embodiment, the MPXV gene probes can be labeled with a donor fluorescent moiety, e.g., a fluorescent dye, and a corresponding acceptor fluorescent moiety, e.g., a quencher. In one embodiment, the probe comprises or consists of a fluorescent moiety and the nucleic acid sequences comprise or consist of SEQ ID NO: 6, 13, 21 and 29.

Designing oligonucleotides to be used as probes can be performed in a manner similar to the design of primers. Embodiments may use a single probe or a pair of probes for detection of the amplification product. Depending on the embodiment, the probe(s) use may comprise at least one label and/or at least one quencher moiety. As with the primers, the probes usually have similar melting temperatures, and the length of each probe must be sufficient for sequence-specific hybridization to occur but not so long that fidelity is reduced during synthesis. Oligonucleotide probes are generally 15 to 30 (e.g., 16, 18, 20, 21, 22, 23, 24, or 25) nucleotides in length.

Constructs containing MPXV nucleic acid molecules can be propagated in a host cell. Prokaryotic hosts may include E. coli, Salmonella typhimurium, Serratia marcescens, and Bacillus subtilis. Eukaryotic hosts include yeasts such as S. cerevisiae, S. pombe, Pichia pastoris, mammalian cells such as COS cells or Chinese hamster ovary (CHO) cells, insect cells, and plant cells such as Arabidopsis thaliana and Nicotiana tabacum. A construct can be introduced into a host cell using any of the techniques commonly known to those of ordinary skill in the art. For example, calcium phosphate precipitation, electroporation, heat shock, lipofection, microinjection, and viral-mediated nucleic acid transfer are common methods for introducing nucleic acids into host cells. In addition, naked DNA can be delivered directly to cells (see, e.g., U.S. Pat. Nos. 5,580,859 and 5,589,466).

U.S. Pat. Nos. 4,683,202, 4,683,195, 4,800,159, and 4,965,188 disclose conventional PCR techniques. PCR typically employs two oligonucleotide primers that bind to a selected nucleic acid template (e.g., DNA or RNA). Primers useful in some embodiments include oligonucleotides capable of acting as points of initiation of nucleic acid synthesis within the described MPXV gene nucleic acid sequences (e.g., SEQ ID NOs: 1-5, 7-12, 14-20, 22-28). A primer can be purified from a restriction digest by conventional methods, or it can be produced synthetically. The primer is preferably single-stranded for maximum efficiency in amplification, but the primer can be double-stranded. Double-stranded primers are first denatured, i.e., treated to separate the strands. One method of denaturing double stranded nucleic acids is by heating.

If the template nucleic acid is double-stranded, it is necessary to separate the two strands before it can be used as a template in PCR. Strand separation can be accomplished by any suitable denaturing method including physical, chemical or enzymatic means. One method of separating the nucleic acid strands involves heating the nucleic acid until it is predominately denatured (e.g., greater than 50%, 60%, 70%, 80%, 90% or 95% denatured). The heating conditions necessary for denaturing template nucleic acid will depend, e.g., on the buffer salt concentration and the length and nucleotide composition of the nucleic acids being denatured, but typically range from about 90° C. to about 105° C. for a time depending on features of the reaction such as temperature and the nucleic acid length. Denaturation is typically performed for about 30 sec to 4 min (e.g., 1 min to 2 min 30 sec, or 1.5 min).

If the double-stranded template nucleic acid is denatured by heat, the reaction mixture is allowed to cool to a temperature that promotes annealing of each primer to its target sequence on the described MPXV gene nucleic acid molecules. The temperature for annealing is usually from about 35° C. to about 65° C. (e.g., about 40° C. to about 60° C.; about 45° C. to about 50° C.). Annealing times can be from about 10 sec to about 1 min (e.g., about 20 sec to about 50 sec; about 30 sec to about 40 sec). The reaction mixture is then adjusted to a temperature at which the activity of the polymerase is promoted or optimized, i.e., a temperature sufficient for extension to occur from the annealed primer to generate products complementary to the template nucleic acid. The temperature should be sufficient to synthesize an extension product from each primer that is annealed to a nucleic acid template, but should not be so high as to denature an extension product from its complementary template (e.g., the temperature for extension generally ranges from about 40° C. to about 80° C. (e.g., about 50° C. to about 70° C.; about 60° C.). Extension times can be from about 10 sec to about 5 min (e.g., about 30 sec to about 4 min; about 1 min to about 3 min; about 1 min 30 sec to about 2 min).

PCR assays can employ MPXV nucleic acid such as RNA or DNA (cDNA). The template nucleic acid need not be purified; it may be a minor fraction of a complex mixture, such as MPXV nucleic acid contained in human cells. MPXV nucleic acid molecules may be extracted from a biological sample by routine techniques such as those described in Diagnostic Molecular Microbiology: Principles and Applications (Persing et al. (eds), 1993, American Society for Microbiology, Washington D.C.). Nucleic acids can be obtained from any number of sources, such as plasmids, or natural sources including bacteria, yeast, viruses, organelles, or higher organisms such as plants or animals.

The oligonucleotide primers (e.g., SEQ ID NOs: 1-5, 7-12, 14-20, 22-28) are combined with PCR reagents under reaction conditions that induce primer extension. For example, chain extension reactions generally include 50 mM KCl, 10 mM Tris-HCl (pH 8.3), 15 mM MgCl₂, 0.001% (w/v) gelatin, 0.5-1.0 μg denatured template DNA, 50 pmoles of each oligonucleotide primer, 2.5 U of Taq polymerase, and 10% DMSO). The reactions usually contain 150 to 320 μM each of dATP, dCTP, dTTP, dGTP, or one or more analogs thereof.

The newly synthesized strands form a double-stranded molecule that can be used in the succeeding steps of the reaction. The steps of strand separation, annealing, and elongation can be repeated as often as needed to produce the desired quantity of amplification products corresponding to the target MPXV gene nucleic acid molecules. The limiting factors in the reaction are the amounts of primers, thermostable enzyme, and nucleoside triphosphates present in the reaction. The cycling steps (i.e., denaturation, annealing, and extension) are preferably repeated at least once. For use in detection, the number of cycling steps will depend, e.g., on the nature of the sample. If the sample is a complex mixture of nucleic acids, more cycling steps will be required to amplify the target sequence sufficient for detection. Generally, the cycling steps are repeated at least about 20 times, but may be repeated as many as 40, 60, or even 100 times. FRET technology (see, for example, U.S. Pat. Nos. 4,996,143, 5,565,322, 5,849,489, and 6,162,603) is based on a concept that when a donor fluorescent moiety and a corresponding acceptor fluorescent moiety are positioned within a certain distance of each other, energy transfer takes place between the two fluorescent moieties that can be visualized or otherwise detected and/or quantitated. The donor typically transfers the energy to the acceptor when the donor is excited by light radiation with a suitable wavelength. The acceptor typically re-emits the transferred energy in the form of light radiation with a different wavelength. In certain systems, non-fluorescent energy can be transferred between donor and acceptor moieties, by way of biomolecules that include substantially non-fluorescent donor moieties (see, for example, U.S. Pat. No. 7,741,467).

In one example, an oligonucleotide probe can contain a donor fluorescent moiety and a corresponding quencher, which may or not be fluorescent, and which dissipates the transferred energy in a form other than light. When the probe is intact, energy transfer typically occurs between the two fluorescent moieties such that fluorescent emission from the donor fluorescent moiety is quenched. During an extension step of a polymerase chain reaction, a probe bound to an amplification product is cleaved by the 5′ to 3′ nuclease activity of, e.g., a Taq Polymerase such that the fluorescent emission of the donor fluorescent moiety is no longer quenched. Exemplary probes for this purpose are described in, e.g., U.S. Pat. Nos. 5,210,015, 5,994,056, and 6,171,785. Commonly used donor-acceptor pairs include the FAM-TAMRA pair. Commonly used quenchers are DABCYL and TAMRA. Commonly used dark quenchers include BlackHole Quenchers™ (BHQ), (Biosearch Technologies, Inc., Novato, Cal.), Iowa Black™, (Integrated DNA Tech., Inc., Coralville, Iowa), BlackBerry™ Quencher 650 (BBQ-650), (Berry & Assoc., Dexter, Mich.).

In another example, two oligonucleotide probes, each containing a fluorescent moiety, can hybridize to an amplification product at particular positions determined by the complementarity of the oligonucleotide probes to the MPXV target nucleic acid sequence. Upon hybridization of the oligonucleotide probes to the amplification product nucleic acid at the appropriate positions, a FRET signal is generated. Hybridization temperatures can range from about 35° C. to about 65° C. for about 10 sec to about 1 min.

Fluorescent analysis can be carried out using, for example, a photon counting epifluorescent microscope system (containing the appropriate dichroic mirror and filters for monitoring fluorescent emission at the particular range), a photon counting photomultiplier system, or a fluorimeter. Excitation to initiate energy transfer, or to allow direct detection of a fluorophore, can be carried out with an argon ion laser, a high intensity mercury (Hg) arc lamp, a fiber optic light source, or other high intensity light source appropriately filtered for excitation in the desired range.

As used herein with respect to donor and corresponding acceptor fluorescent moieties “corresponding” refers to an acceptor fluorescent moiety having an absorbance spectrum that overlaps the emission spectrum of the donor fluorescent moiety. The wavelength maximum of the emission spectrum of the acceptor fluorescent moiety should be at least 100 nm greater than the wavelength maximum of the excitation spectrum of the donor fluorescent moiety.

Accordingly, efficient non-radiative energy transfer can be produced there between. Fluorescent donor and corresponding acceptor moieties are generally chosen for (a) high efficiency Forster energy transfer; (b) a large final Stokes shift (>100 nm); (c) shift of the emission as far as possible into the red portion of the visible spectrum (>600 nm); and (d) shift of the emission to a higher wavelength than the Raman water fluorescent emission produced by excitation at the donor excitation wavelength. For example, a donor fluorescent moiety can be chosen that has its excitation maximum near a laser line (for example, Helium-Cadmium 442 nm or Argon 488 nm), a high extinction coefficient, a high quantum yield, and a good overlap of its fluorescent emission with the excitation spectrum of the corresponding acceptor fluorescent moiety. A corresponding acceptor fluorescent moiety can be chosen that has a high extinction coefficient, a high quantum yield, a good overlap of its excitation with the emission of the donor fluorescent moiety, and emission in the red part of the visible spectrum (>600 nm).

Representative donor fluorescent moieties that can be used with various acceptor fluorescent moieties in FRET technology include fluorescein, Lucifer Yellow, B-phycoerythrin, 9-acridineisothiocyanate, Lucifer Yellow VS, 4-acetamido-4′-isothio-cyanatostilbene-2,2′-disulfonic acid, 7-diethylamino-3-(4′-isothiocyanatophenyl)-4-methylcoumarin, succinimdyl 1-pyrenebutyrate, and 4-acetamido-4′-isothiocyanatostilbene-2,2′-disulfonic acid derivatives. Representative acceptor fluorescent moieties, depending upon the donor fluorescent moiety used, include LC Red 640, LC Red 705, Cy5, Cy5.5, Lissamine rhodamine B sulfonyl chloride, tetramethyl rhodamine isothiocyanate, rhodamine x isothiocyanate, erythrosine isothiocyanate, fluorescein, diethylenetriamine pentaacetate, or other chelates of Lanthanide ions (e.g., Europium, or Terbium). Donor and acceptor fluorescent moieties can be obtained, for example, from Molecular Probes (Junction City, Oreg.) or Sigma Chemical Co. (St. Louis, Mo.).

The donor and acceptor fluorescent moieties can be attached to the appropriate probe oligonucleotide via a linker arm. The length of each linker arm is important, as the linker arms will affect the distance between the donor and acceptor fluorescent moieties. The length of a linker arm can be the distance in Angstroms (Å) from the nucleotide base to the fluorescent moiety. In general, a linker arm is from about 10 Å to about 25 Å. The linker arm may be of the kind described in WO 84/03285. WO 84/03285 also discloses methods for attaching linker arms to a particular nucleotide base, and also for attaching fluorescent moieties to a linker arm.

An acceptor fluorescent moiety, such as an LC Red 640, can be combined with an oligonucleotide which contains an amino linker (e.g., C6-amino phosphoramidites available from ABI (Foster City, Calif.) or Glen Research (Sterling, VA)) to produce, for example, LC Red 640-labeled oligonucleotide. Frequently used linkers to couple a donor fluorescent moiety such as fluorescein to an oligonucleotide include thiourea linkers (FITC-derived, for example, fluorescein-CPG's from Glen Research or ChemGene (Ashland, Mass.)), amide-linkers (fluorescein-NHS-ester-derived, such as CX-fluorescein-CPG from BioGenex (San Ramon, Calif.)), or 3′-amino-CPGs that require coupling of a fluorescein-NHS-ester after oligonucleotide synthesis.

IIa. Detection of Monkeypox Virus

The present disclosure provides methods for detecting the presence or absence of Monkeypox Virus (MPXV) in a biological or non-biological sample. Methods provided avoid problems of sample contamination, false negatives, and false positives. The methods include performing at least one cycling step that includes amplifying a portion of the MPXV F3L gene and/or the MPXV B21R gene target nucleic acid molecules from a sample using pairs of F3L gene and/or B21R gene primers, and a FRET detecting step. Multiple cycling steps are performed, preferably in a thermocycler. Methods can be performed using the F3L gene and/or B21R gene primers and probes to detect the presence of MPXV, and the detection of MPXV F3L gene and/or the MPXV B21R gene indicates the presence of MPXV in the sample.

As described herein, amplification products can be detected using labeled hybridization probes that take advantage of FRET technology. One FRET format utilizes TaqMan® technology to detect the presence or absence of an amplification product, and hence, the presence or absence of MPXV. TaqMan® technology utilizes one single-stranded hybridization probe labeled with, e.g., one fluorescent dye and one quencher, which may or may not be fluorescent. When a first fluorescent moiety is excited with light of a suitable wavelength, the absorbed energy is transferred to a second fluorescent moiety according to the principles of FRET. The second fluorescent moiety is generally a quencher molecule. During the annealing step of the PCR reaction, the labeled hybridization probe binds to the target DNA (i.e., the amplification product) and is degraded by the 5′ to 3′ nuclease activity of, e.g., the Taq Polymerase during the subsequent elongation phase. As a result, the fluorescent moiety and the quencher moiety become spatially separated from one another. As a consequence, upon excitation of the first fluorescent moiety in the absence of the quencher, the fluorescence emission from the first fluorescent moiety can be detected. By way of example, an ABI PRISM® 7700 Sequence Detection System (Applied Biosystems) uses TaqMan® technology, and is suitable for performing the methods described herein for detecting the presence or absence of MPXV in the sample.

Molecular beacons in conjunction with FRET can also be used to detect the presence of an amplification product using the real-time PCR methods. Molecular beacon technology uses a hybridization probe labeled with a first fluorescent moiety and a second fluorescent moiety. The second fluorescent moiety is generally a quencher, and the fluorescent labels are typically located at each end of the probe. Molecular beacon technology uses a probe oligonucleotide having sequences that permit secondary structure formation (e.g., a hairpin). As a result of secondary structure formation within the probe, both fluorescent moieties are in spatial proximity when the probe is in solution. After hybridization to the target nucleic acids (i.e., amplification products), the secondary structure of the probe is disrupted and the fluorescent moieties become separated from one another such that after excitation with light of a suitable wavelength, the emission of the first fluorescent moiety can be detected.

Another common format of FRET technology utilizes two hybridization probes. Each probe can be labeled with a different fluorescent moiety and are generally designed to hybridize in close proximity to each other in a target DNA molecule (e.g., an amplification product). A donor fluorescent moiety, for example, fluorescein, is excited at 470 nm by the light source of the LightCycler® Instrument. During FRET, the fluorescein transfers its energy to an acceptor fluorescent moiety such as LightCycler®-Red 640 (LC Red 640) or LightCycler®-Red 705 (LC Red 705). The acceptor fluorescent moiety then emits light of a longer wavelength, which is detected by the optical detection system of the LightCycler® instrument. Efficient FRET can only take place when the fluorescent moieties are in direct local proximity and when the emission spectrum of the donor fluorescent moiety overlaps with the absorption spectrum of the acceptor fluorescent moiety. The intensity of the emitted signal can be correlated with the number of original target DNA molecules (e.g., the number of MPXV genomes). If amplification of MPXV target nucleic acid occurs and an amplification product is produced, the step of hybridizing results in a detectable signal based upon FRET between the members of the pair of probes.

Generally, the presence of FRET indicates the presence of MPXV in the sample, and the absence of FRET indicates the absence of MPXV in the sample. Inadequate specimen collection, transportation delays, inappropriate transportation conditions, or use of certain collection swabs (calcium alginate or aluminum shaft) are all conditions that can affect the success and/or accuracy of a test result, however. Using the methods disclosed herein, detection of FRET within, e.g., 45 cycling steps is indicative of an MPXV infection.

Representative biological samples that can be used in practicing the methods include, but are not limited to blood, plasma, serum, liver samples, dermal swabs, nasal swabs, lesion swabs, blood cultures, skin, and soft tissue infections. Collection and storage methods of biological samples are known to those of skill in the art. Biological samples can be processed (e.g., by nucleic acid extraction methods and/or kits known in the art) to release MPXV nucleic acid or in some cases, the biological sample can be contacted directly with the PCR reaction components and the appropriate oligonucleotides.

Melting curve analysis is an additional step that can be included in a cycling profile. Melting curve analysis is based on the fact that DNA melts at a characteristic temperature called the melting temperature (Tm), which is defined as the temperature at which half of the DNA duplexes have separated into single strands. The melting temperature of a DNA depends primarily upon its nucleotide composition. Thus, DNA molecules rich in G and C nucleotides have a higher Tm than those having an abundance of A and T nucleotides. By detecting the temperature at which signal is lost, the melting temperature of probes can be determined. Similarly, by detecting the temperature at which signal is generated, the annealing temperature of probes can be determined. The melting temperature(s) of the F3L gene and B21R gene probes from the respective amplification products can confirm the presence or absence of MPXV in the sample.

Within each thermocycler run, control samples can be cycled as well. Positive control samples can amplify target nucleic acid control template (other than described amplification products of target genes) using, for example, control primers and control probes. Positive control samples can also amplify, for example, a plasmid construct containing the target nucleic acid molecules. Such a plasmid control can be amplified internally (e.g., within the sample) or in a separate sample run side-by-side with the patients' samples using the same primers and probe as used for detection of the intended target. Such controls are indicators of the success or failure of the amplification, hybridization, and/or FRET reaction. Each thermocycler run can also include a negative control that, for example, lacks target template DNA. Negative control can measure contamination. This ensures that the system and reagents would not give rise to a false positive signal. Therefore, control reactions can readily determine, for example, the ability of primers to anneal with sequence-specificity and to initiate elongation, as well as the ability of probes to hybridize with sequence-specificity and for FRET to occur.

In an embodiment, the methods include steps to avoid contamination. For example, an enzymatic method utilizing uracil-DNA glycosylase is described in U.S. Pat. Nos. 5,035,996, 5,683,896 and 5,945,313 to reduce or eliminate contamination between one thermocycler run and the next. Conventional PCR methods in conjunction with FRET technology can be used to practice the methods. In one embodiment, a LightCycler® instrument is used. The following patent applications describe real-time PCR as used in the LightCycler® technology: WO 97/46707, WO 97/46714, and WO 97/46712. In another embodiment, the cobas® 5800/6800/8800 systems as described in U.S. Pat. Nos. 8,476,015 and 9,034,575 are used to practice the current methods.

The LightCycler® can be operated using a PC workstation and can utilize a Windows NT operating system. Signals from the samples are obtained as the machine positions the capillaries sequentially over the optical unit. The software can display the fluorescence signals in real-time immediately after each measurement. Fluorescent acquisition time is 10-100 milliseconds (msec). After each cycling step, a quantitative display of fluorescence vs. cycle number can be continually updated for all samples. The data generated can be stored for further analysis.

As an alternative to FRET, an amplification product can be detected using a double-stranded DNA binding dye such as a fluorescent DNA binding dye (e.g., SYBR® Green or SYBR® Gold (Molecular Probes)). Upon interaction with the double-stranded nucleic acid, such fluorescent DNA binding dyes emit a fluorescence signal after excitation with light at a suitable wavelength. A double-stranded DNA binding dye such as a nucleic acid intercalating dye also can be used. When double-stranded DNA binding dyes are used, a melting curve analysis is usually performed for confirmation of the presence of the amplification product.

It is understood that the embodiments of the present disclosure are not limited by the configuration of one or more commercially available instruments.

IIb. Articles of Manufacture/Kits

Embodiments of the present disclosure further provide for articles of manufacture or kits to detect MPXV. An article of manufacture can include primers and probes used to detect MPXV, together with suitable packaging materials. Representative primers and probes for detection of MPXV are capable of hybridizing to MPXV target nucleic acid molecules (e.g. the MPXV F3L and B21R genes). In addition, the kits may also include suitably packaged reagents and materials needed for DNA immobilization, hybridization, and detection, such solid supports, buffers, enzymes, and DNA standards. Methods of designing primers and probes are disclosed herein, and representative examples of primers and probes that amplify and hybridize to MPXV target nucleic acid molecules are provided.

Articles of manufacture can also include one or more fluorescent moieties for labeling the probes or, alternatively, the probes supplied with the kit can be labeled. For example, an article of manufacture may include a donor and/or an acceptor fluorescent moiety for labeling the MPXV F3L and/or the B21R gene probes. Examples of suitable FRET donor fluorescent moieties and corresponding acceptor fluorescent moieties are provided above.

Articles of manufacture can also contain a package insert or package label that have instructions thereon for using the MPXV target gene primers and probes to detect MPXV in a sample. Articles of manufacture may additionally include reagents for carrying out the methods disclosed herein (e.g., buffers, polymerase enzymes, co-factors, or agents to prevent contamination). Such reagents may be specific for one of the commercially available instruments described herein. Embodiments of the present disclosure will be further described in the following examples, which do not limit the scope of the invention described in the claims.

### EXAMPLES

The following examples and figures are provided to aid the understanding of the subject matter, the true scope of which is set forth in the appended claims. It is understood that modifications can be made in the procedures set forth without departing from the spirit of the invention.

### Example 1: MPXV Gene Targets

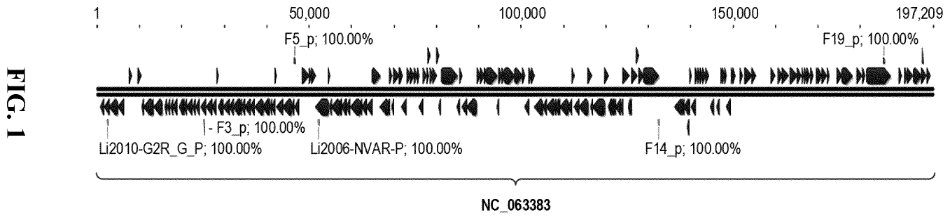

FIG. 1 shows the relative locations of the primers and probes used for amplification and detection of the MPXV gene targets relative to GenBank Accession No. NC_063383, a full genomic sequence of a clade II strain published by CDC. In this figure, F3 refers to the C2L gene region, F5 refers to the F3L gene region, F14 refers to the intragenic non-coding sequence, and F19 refers to the B21R gene region.

### Example 2: PCR Experimental Conditions

Real-time PCR detection of the MPXV target genes were performed using the cobas® 6800/8800 systems (Roche Molecular Systems, Inc., Pleasanton, CA). The final concentrations of the amplification reagents are shown below:

TABLE V
PCR Amplification Reagents

| Master Mix Component | Final Conc (50 uL) | |
|---|---|---|
| DMSO | 5.2 | % |
| NaN3 | 0.030 | % |
| Potassium acetate | 120.0 | mM |
| Glycerol | 2.9 | % |
| Tween 20 | 0.02 | % |
| EDTA | 42.2 | uM |
| Tricine | 60.0 | mM |
| Aptamer | 0.222 | μM |
| UNG Enzyme | 10.0 | U |
| Z05-SP-PZ Polymerase | 45.0 | U |
| dATP | 400.0 | μM |
| dCTP | 400.0 | μM |
| dGTP | 400.0 | μM |
| dUTP | 800.0 | μM |
| Forward primer oligonucleotides | 0.30 | μM |
| Reverse primer oligonucleotides | 0.30 | μM |
| Probe oligonucleotides | 0.15 | μM |
| Manganese Acetate | 3.30-3.80 | mM |

The following table shows the typical thermoprofile used for PCR amplification reaction:

TABLE VI
PCR Thermoprofile

| Program | Target | Acquisition | Hold | Ramp Rate | | Analysis |
|---|---|---|---|---|---|---|
| Name | (° C.) | Mode | (hh:mm:ss) | (° C./s) | Cycles | Mode |
| Pre-PCR | 50 | None | 00:02:00 | 4.4 | 1 | None |
| | 94 | None | 00:00:05 | 4.4 | | |
| | 55 | None | 00:02:00 | 2.2 | | |
| | 60 | None | 00:06:00 | 4.4 | | |
| | 65 | None | 00:04:00 | 4.4 | | |
| 1st | 95 | None | 00:00:05 | 4.4 | 5 | Quantification |
| Measurement | 55 | Single | 00:00:30 | 2.2 | | |
| 2nd | 91 | None | 00:00:05 | 4.4 | 45 | Quantification |
| Measurement | 58 | Single | 00:00:25 | 2.2 | | |
| Cooling | 40 | None | 00:02:00 | 2.2 | 1 | None |

The Pre-PCR program comprised initial denaturing and incubation at 55° C., 60° C. and 65° C. for reverse transcription of RNA templates. Incubating at three temperatures combines the advantageous effects that at lower temperatures slightly mismatched target sequences (such as genetic variants of an organism) are also transcribed, while at higher temperatures the formation of RNA secondary structures is suppressed, thus leading to a more efficient transcription. PCR cycling was divided into two measurements, wherein both measurements apply a one-step setup (combining annealing and extension). The first 5 cycles at 55° C. allow for an increased inclusivity by pre-amplifying slightly mismatched target sequences, whereas the 45 cycles of the second measurement provide for an increased specificity by using an annealing/extension temperature of 58° C.

### Example 3a: Linearity Study

Plasmids were used for linearity studies. Plasmids containing amplicon sequences of monkeypox assay were synthesized and manufactured by Integrated DNA Technology (IDT). Plasmids were linearized by restriction enzyme and the copy numbers were determined by droplet digital PCR (ddPCR, BioRad).

For the linearity study, a 10-fold serial dilutions of plasmid templates at the concentrations of 10-10\hat{ }9 copies/mL of matrix were tested with 3 replicates per condition. Two types of matrices were tested-Negative Human Plasma (NHP) and cobas® PCR media (Roche Molecular Systems) with skin swabs samples. Plasmid DNA was spiked into matrix and went through sample prep process and generate eluates on the cobas® 6800 instrument (Roche Molecular Systems). The eluates containing plasmid DNA were subsequently mixed with PCR mix containing monkeypox assays and underwent polymerase chain reaction in the same instrument. Results were analyzed with Colibri and JMP software. For linearity study, Slope, the square of the correlation coefficient (R squared) were calculated with Excel, by Ct values against log values of template input levels.

### Example 3b: Limit of Detection (LoD) Study

Viral genomic DNA was used for Limit of Detection (LoD) studies. Genomic DNA of Monkeypox Virus, USA-2003 (NR-4928) was acquired from BEI Resources. Copy number viral gDNA was determined by droplet digital PCR (ddPCR, BioRad).

For LoD study, viral genomic DNA were diluted for 5 levels and with 9 to 20 replicates for each level. Viral gDNA were spiked into matrix and went through sample prep process and generated eluates on the cobas® 6800 instrument. Two types of matrices were tested—Negative Human Plasma (NHP) and cobas® PCR media with skin swabs samples. The eluates containing viral gDNA were subsequently mixed with PCR mix containing monkeypox assays and underwent polymerase chain reaction in the same instrument.

For LoD study, the number of reactive and non-reaction samples at each input level was analyzed by Probit function in JMP software. LoD as well as lower and upper limit of LoD at 95% Confidence Interval was predicted by Probit analysis.

### Example 4: Experimental Results

A prototype MPXV PCR assay was performed using the set of primers and probes shown in TABLE VII.

TABLE VII
MPXV Assay Primers and Probes

| SEQ ID NO | NAME | DESCRIPTION | SEQUENCE |
|---|---|---|---|
| 7 | MKPX5- | F3L Forward primer | CTCTCATTGATTTTTCGCGGG |
| | F1_A | | AT<t_BB_dA> |
| 11 | MKPX5- | F3L Reverse primer | ACGACAATGGATGCTGATAC |
| | R1_A | | A<t_BB_dC> |
| 13 | MKPX5- | F3L Probe | <FAM_Thr>TAGCATC<BHQ_2> |
| | FAM | | AGCATCAGAATCTGTAGGC |
| | | | CGT<Spc_C3> |
| 25 | MKPX19- | B21R Forward | GACCCACGAGTTATAACAAA |
| | F4_A | primer | GTCA<t_BB_dA> |
| 26 | MKPX19- | B21R Reverse primer | CCAAAATGAATTAGGGTTGT |
| | R1_A | | CATATCC<t_BB_dA> |
| 29 | MKPX19- | B21R Probe | <FAM_Thr>ACCCATT<BHQ_2> |
| | FAM | | TTCCTGCTCCATTGAGTGTA |
| | | | AAATTCATG<Spc_C3> |

<t_BB_dA> = t-butyl-benzyl-dA; <t_BB_dC> = t-butyl-benzyl-dC; <FAM_Thr> = FAM dye; <BHQ_2> = Quencher; < Spc_C3> = 3′ blocker

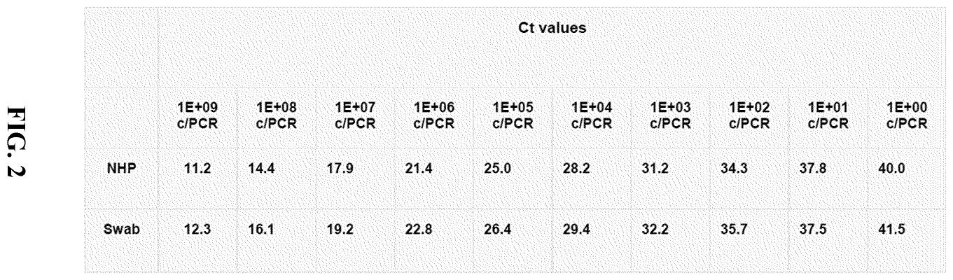

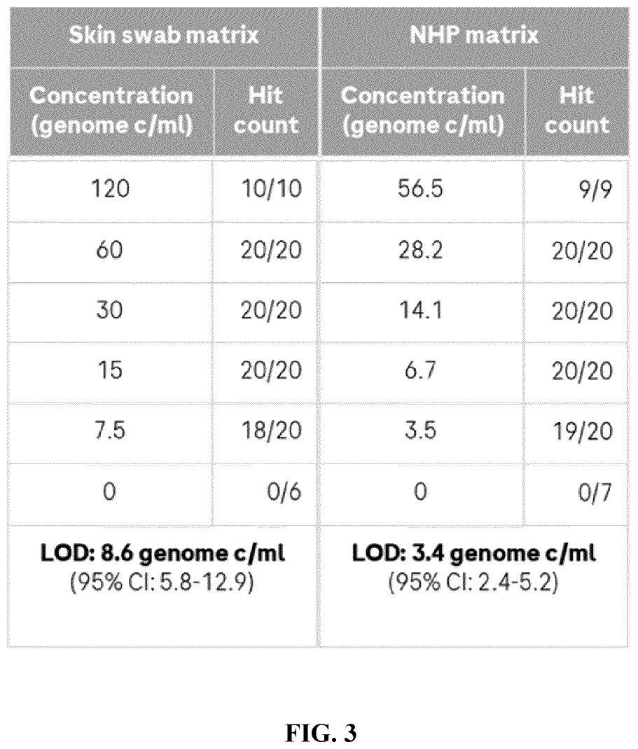

Results of the linearity study are shown in FIG. 2 and demonstrates good linearity between 10-10\hat{ }9 copies of plasmid per ml of matrix. Results of the limit of detection (LoD) study are shown in FIG. 3 and demonstrated LoD of 8.6 copies/ml (95% CI: 5.8-12.9) for spiked skin swab matrix samples and 3.4 copies/ml (2.4-5.2) for spiked NHP matrix samples.

### Example 5: Analytical Sensitivy/Limit of Detection (LoD)

Limit of Detection (LoD) studies determine the lowest detectable concentration of MPXV at which greater or equal to 95% of all (true positive) replicates test positive. To determine the LoD, a heat-inactivated cultured virus of an isolate from a Slovenian patient (isolate 225/22 Slovenia ex Gran Canaria strain Slovenia_MPXV-1_2022, European Virus Archive-global (EVAg), clade IIb, lineage B.1, 10E+06 TCID₅₀/mL before heat inactivation, 5.79E+09 cp/mL using droplet digital PCR determination) was serially diluted in pooled negative clinical specimen, negative for MPXV. A total of 7 concentration levels, with 2-fold serial dilutions between the levels, were tested with a total of 42 replicates per concentration, with an additional 42 replicates of a blank (unspiked pool). As shown below in TABLE VIII, the concentration level with observed hit rates greater than or equal to 95% were 0.01 TCID₅₀/mL or 57 cp/mL for MPXV (Target 1). Beta-globin (Target 2) was positive for all tested replicates.

TABLE VIII
LoD Determination

| | | | Hit Rate [%] | | Mean Ct¹ | |
|---|---|---|---|---|---|---|
| | | Total | | Target 2 | | Target 2 |
| Concentration | Concentration | valid | Target 1 | (β- | Target 1 | (β - |
| [TCID50/mL] | [cp/mL] | results | (MPXV) | globin) | (MPXV) | globin) |
| 4.00E−02 | 228.0 | 42 | 100.0 | 100.0 | 33.81 | 27.00 |
| 2.00E−02 | 114.0 | 42 | 100.0 | 100.0 | 34.73 | 26.96 |
| 1.00E−02 | 57.0 | 42 | 97.6 | 100.0 | 35.92 | 27.08 |
| 5.00E−03 | 28.5 | 42 | 92.9 | 100.0 | 37.12 | 27.07 |
| 2.50E−03 | 14.3 | 42 | 71.4 | 100.0 | 37.35 | 26.94 |
| 1.25E−03 | 7.1 | 42 | 33.3 | 100.0 | 37.85 | 26.99 |
| 6.25E−04 | 3.6 | 42 | 14.3 | 100.0 | 37.94 | 27.05 |
| 0.0 (blank) | 0.0 (blank) | 42 | 0.0 | 100.0 | n/a | 27.07 |

Also, as shown in TABLE IX, the Probit predicted 95% hit rate was 0.0064 TCID₅₀/mL or 36.5 cp/mL for MPXV (Target 1).

TABLE IX
Probit Predicted 95% Hit Rates

| | Probit Predicted | Probit Predicted |
|---|---|---|
| | 95% Hit Rate | 95% Hit Rate |
| Strain | [TCID50/mL] | [cp/mL] |
| Slovenia_MPXV- | 6.35E−03 | 36.5 |
| 1_2022 | (95% CI: 4.82E−03- | (95% CI: 27.4-54.2) |
| | 9.50E−03) | |

### Example 6: In Silico Inclusivity Study

Inclusivity was assessed in silico using MPXV genomes submitted to the NCBI (taxa ID 10244) and GISAID repositories. While both repositories were assessed individually, overlap between the two repositories can be expected. Out of the 623 available sequences on NCBI that cover the F3L gene-targeted region, 606 sequences (97.3%) show 100% identity with the amplification product generated by the primers of SEQ ID NO: 7 and SEQ ID NO: 11. Fifteen sequences collected during a non-human primate study in Cote d′Ivoire in 2012 (example KJ136820.1) show a single mutation in the reverse primer with minimal expected impact on the F3L gene assay performance. The remaining two sequences (example FV537352.1) show at least 5 mutations in the forward primer, reverse primer and probe sequences that would cause failure of the F3L gene assay. However, these two failing sequences are derived from modified nucleic acid of unknown origin and are therefore of no concern. Of note, all sequences with one or more mutations in the F3L gene-targeted region did not carry any mutation in the B21R gene region that is also targeted in the multiplexed prototype. Out of the 695 available sequences on GISAID that cover the F3L gene-targeted region, all 695 sequences (100%) show 100% identity with the sequences of SEQ ID NOs: 7, 11 and 13.

Out of the 624 available sequences on NCBI that cover the B21R gene-targeted region, 622 sequences (99.7%) show 100% identity with the sequences of SEQ ID NOs: 25, 26 and 29. Two sequences show a mutation in the probe-binding region (13th of 36 probe nucleotides) with minimal expected impact on assay performance. Out of the 696 available sequences on GISAID that cover the B21R gene-targeted region, 695 sequences (99.9%) show 100% identity with the sequences of SEQ ID NOs: 25, 26 and 29. A single sequence (EPI_ISL_13472080) carried a mutation in the forward primer that is expected to have a minimal impact on assay performance.

### Example 7: Clinical Performance

A clinical agreement study at one external site (Labcorp Central Laboratory Services, Burlington, North Carolina, USA, hereinafter, “test site”) was conducted to compare the MPXV Assay of the present invention and a comparator test [Non-variola Orthopoxvirus Real-time PCR Primer and Probe Set, CDC (K222558)] using fresh (never frozen) natural leftover de-identified clinical specimens from routine clinical testing. The comparator test was performed first for routine patient management according to the cleared instructions for use with two modifications previously communicated by FDA to the CDC as areas under and within enforcement discretion. The modifications to the CDC Non-variola Orthopoxvirus Real-time PCR Primer and Probe Set were (i) the use of the MagNA Pure 96 System (Roche Molecular Systems, Inc., Pleasanton, CA) for nucleic acid extraction and (ii) the use of the Applied Biosystems QuantStudio 7 Flex Real-Time PCR System as an additional testing platform.

After completion of the routine testing of clinical specimens, positive and negative specimens per the comparator method that were non-frozen and were transported as wet swabs in Copan Universal Transport Media (UTM) were selected for MPXV testing. The test site selected the first 30 negative on the first day of testing. Additional negative samples were included on subsequent runs in order to have both positive and negative samples in most runs. The test site selected the first 30 positive samples over a four-day period.

In addition, after completion of the 30 positive samples, five additional positive specimens were identified and used to prepare contrived low positive specimens based on the comparator method. Specimens were serially diluted into unique negative lesion specimens and re-tested on the comparator method to identify a contrived specimens with 31<Ct<37. The low positive specimens were subsequently tested on the MPXV assay of the present invention and the results of this study can be found on TABLE X.

TABLE X
Results of the Clinical Performance Study

| | | CDC reference method | | |
|---|---|---|---|---|
| | | Positive | Negative | Total |
| cobas ® | Positive | 35 | 0 | 35 |
| MPXV | Negative | 0 | 47 | 47 |
| | Total | 35 | 47 | 82 |

| | | 95% Score |
|---|---|---|
| Percent | | Confidence |
| Agreement | Result (%) | Interval (%) |
| PPA | 100 (35/35) | 90.1-100.0 |
| NPA | 100 (47/47) | 92.4-100.0 |

The contrived low positive samples, in conjunction with the two naturally-occuring low positive samples (Ct's 31.35 and 35.44 in the CDC reference test) were used to assess the performance of the MPXV Assay of the present invention in low titer specimens and the results are seen in TABLE XI.

TABLE XI
Clinical Evaluation of Low Positive Samples

| | | Target 1 | | Target 2 | |
|---|---|---|---|---|---|
| | | (MPXV) | | (beta-globin) | |
| | | % positive | Target 1 | % positive | Target 2 |
| | | (two-sided 95% | Mean | (two-sided 95% | Mean |
| Sample | N | Score CI) | Ct | Score CI) | Ct |
| Ct 31 to Ct | 7 | 100 | 29.8 | 100 | 29.3 |
| 37 with CDC | | (64.6-100) | | (64.6-100) | |
| reference | | | | | |
| method | | | | | |

### Example 8: In-Silico Exclusivity Study

Desired exclusivity to other orthopoxviruses was taken into account during in silico assay design with primarily the probe sequence being highly specific to MPXV and exclusive to other orthopoxviruses. The top three cowpox gene fragments with the highest potential for amplification (KC813493, KY569022, MK035758) and one camelpox gene fragment (MZ300860) were also tested as gBlocks (synthetic double stranded DNA fragments) at 1E4, 1E6 and 5E8 copies per reaction in triplicate. No signal was detected in any of these reactions. Laboratory testing of organisms that may cause a clinical spectrum similar to that of MPXV was also performed in a clean background. HSV1 gDNA (˜6E8 genomes/PCR), HSV2 gDNA (˜4E8 genomes/PCR), VZV gDNA (8E8 genomes/PCR), Treponema gDNA (ATCC TSD-166, 3.3 ng/PCR), and cowpox virus gDNA (BEI NR-2641, 5E4 copies/PCR) all failed to yield any positive results when tested in triplicate. A skin microbiome whole cell mix DNA (ATCC, MSA-2005) tested at 5 ng/PCR also failed to yield any positive results.

In addition, analysis of low MPXV positives and negative controls were sent for sequencing to identify products that may have been amplified but were not detected by the probes (‘CACTUS analysis’, a Roche proprietary analysis to Connect, Align/Annotate, Cluster Target Unknown Sequences through next generation sequencing). This did not reveal any high level non-specific amplification events, but identified low-level Burkholderia contaminans and Cutibacterium acnes amplicons in skin swab matrix samples, and Serratia proteamaculans, Shewanella baltica and Pseudomonas lundensis and other Pseudomonas sp. in negative human plasma samples. These organisms would thus be good candidates for more extensive exclusivity testing during further development of the MPXV prototype assay.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A method for detecting at least two target nucleic acids of Monkeypox Virus (MPXV) in a sample, the method comprising:
(a) providing a sample;
(b) performing an amplification step comprising contacting the sample with at least two sets of oligonucleotide primers to produce amplification products if the at least two target nucleic acids of MPXV are present in the sample;
(c) performing a hybridization step comprising contacting the amplification products with at least two oligonucleotide probes; and
(d) performing a detection step comprising detecting the presence or absence of the amplification products, wherein the presence of at least one of the amplification products is indicative of the presence of MPXV in the sample and wherein the absence of the amplification products is indicative of the absence of MPXV in the sample; and
wherein the at least two target nucleic acids of MPXV are selected from the group consisting of a gene encoding a serine protease inhibitor-like protein (C2L gene), a gene encoding a putative double-stranded RNA binding protein (F3L gene), an intergenic non-coding sequence between the A25R and A26L genes (INCS), and a gene encoding an immunogenic membrane-associated glycoprotein (B21R gene).

2. The method of claim 1, wherein the at least two target nucleic acids of MPXV are the F3L gene and the B21R gene.

3. The method of claim 2, wherein the at least two sets of oligonucleotide primers and the at least two oligonucleotide probes comprise:
(i) a first set of oligonucleotide primers for amplification of the F3L gene target nucleic acid comprising a forward primer comprising a nucleic acid sequence selected from the group consisting of SEQ ID NOs: 7-10, or any combination of forward primers each comprising a nucleic acid sequence selected from the group consisting of SEQ ID NOs: 7-10; and a reverse primer comprising a nucleic acid sequence selected from the group consisting of SEQ ID NOs: 11-12, or a combination thereof; and a first probe or a first set of probes for detection of an amplification product of the F3L gene target nucleic acid comprising a nucleic acid sequence of SEQ ID NO: 13, or a complement thereof; and
(ii) a second set of oligonucleotide primers for amplification of the B21R gene target nucleic acid comprising a forward primer comprising a nucleic acid sequence selected from the group consisting of SEQ ID NOs: 22-25, or any combination of forward primers each comprising a nucleic acid sequence selected from the group consisting of SEQ ID NOs: 22-25; and a reverse primer comprising a nucleic acid sequence selected from the group consisting of SEQ ID NOs: 26-28, or any combination of reverse primers each comprising a nucleic acid sequence selected from the group consisting of SEQ ID NOs: 22-25; and a second probe or a second set of probes for detection of an amplification product of the B21R gene target nucleic acid comprising a nucleic acid sequence of SEQ ID NO: 29, or a complement thereof.

4. The method of claim 3, wherein the first set of oligonucleotide primers for amplification of the F3L gene target nucleic acid comprises a forward primer comprising or consisting of a nucleic acid sequence of SEQ ID NO: 7 and a reverse primer comprising or consisting of a nucleic acid sequence of SEQ ID NO: 11, and the second set of oligonucleotide primers of amplification of the B21R gene target nucleic acid comprises a forward primer comprising or consisting of a nucleic acid sequence of SEQ ID NO: 25 and a reverse primer comprising or consisting of a nucleic acid sequence of SEQ ID NO: 26.

5. The method of claim 1, wherein:
the hybridization step comprises contacting the amplification products with the at least two oligonucleotide probes that are each labeled with a donor fluorescent moiety and a corresponding acceptor moiety; and
the detection step comprises detecting the presence or absence of fluorescence resonance energy transfer (FRET) between the donor fluorescent moiety and the acceptor moiety of the probes, wherein the presence or absence of fluorescence is indicative of the presence or absence of MPXV in the sample.

6. The method of claim 5, wherein the at least two oligonucleotide probes are each labeled with the same donor fluorescent moiety.

7. The method of claim 5, wherein the at least two oligonucleotide probes are each labeled with a different donor fluorescent moiety.

8. The method of claim 1, the 7, wherein the amplification step employs a polymerase enzyme having 5′ to 3′ nuclease activity.

9. The method of claim 1, wherein the sample is a biological sample selected from a skin swab, a lesion swab, and plasma.

10. (canceled)

11. A method for detecting Monkeypox Virus (MPXV) in a sample, the method comprising:
(a) performing an amplification step comprising contacting the sample with a one or more forward oligonucleotide primers and one or more reverse oligonucleotide primers specifically hybridizing to the MPXV F3L gene to produce amplification products of the F3L gene if MPXV is present in the sample; and one or more forward oligonucleotide primers and one or more reverse oligonucleotide primers specifically hybridizing to the MPXV B21R gene to produce amplification products of the B21R gene if MPXV is present in the sample;
(b) performing a hybridization step comprising contacting the F3L gene amplification products with one or more detectable oligonucleotide probes specifically hybridizing to the F3L gene amplification products and contacting the B21R gene amplification products with one or more detectable oligonucleotide probes specifically hybridizing to the B21R gene amplification products; and
(c) detecting the presence or absence of the F3L gene amplification products and/or the B21R gene amplification products, wherein the presence of either the F3L gene amplification products or the B21R amplification products or both amplification products is indicative of the presence of MPXV in the sample and wherein the absence of both the F3L gene amplification products and the B21R amplification products is indicative of the absence of MPXV in the sample;
wherein one of the one or more F3L gene forward oligonucleotide primers comprises or consists of a nucleic acid sequence selected from SEQ ID NOs: 7-10, or the one or more F3L gene forward oligonucleotide primers comprise any combination of forward primers each comprising or consisting of a nucleic acid sequence selected from the group consisting of SEQ ID NOs: 7-10; and one of the one or more F3L gene reverse oligonucleotide primers comprises or consists of a nucleic acid sequence selected from SEQ ID NOs: 11-12, or the one or more F3L gene reverse oligonucleotide primers comprise two reverse primers comprising or consisting of a nucleic acid sequence of SEQ ID NOs: 11 and 12; and one of the one or more detectable F3L gene oligonucleotide probes comprises or consists of a nucleic acid sequence of SEQ ID NO: 13, or a complement thereof; and
wherein one of the one or more B21R gene forward oligonucleotide primers comprises or consists of a nucleic acid sequence selected from SEQ ID NOs: 22-25, or the one or more B21R gene forward oligonucleotide primers comprise any combination of forward primers each comprising or consisting of a nucleic acid sequence selected from the group consisting of SEQ ID NOs: 22-25; and one of the one or more B21R gene reverse oligonucleotide primers comprises or consists of a nucleic acid sequence selected from SEQ ID NOs: 26-28, or the one or more B21R gene reverse oligonucleotide primers comprise any combination of reverse primers each comprising or consisting of a nucleic acid sequence selected from the group consisting of SEQ ID NOs: 26-28; and one of the one or more detectable B21R gene oligonucleotide probes comprises or consists of a nucleic acid sequence of SEQ ID NO: 29, or a complement thereof.

12. The method of claim 11, wherein one of the one or more F3L gene forward oligonucleotide primers comprises or consists of the nucleic acid sequence of SEQ ID NO: 7, and one of the one or more F3L gene reverse oligonucleotide primers comprises or consists of the nucleic acid sequence of SEQ ID NO: 11.

13. The method of claim 11, wherein one the one or more B21R gene forward oligonucleotide primers comprises or consists of the nucleic acid sequence of SEQ ID NO: 25, and one of the one or more B21R gene reverse oligonucleotide primers comprises or consists of the nucleic acid sequence of SEQ ID NO: 26.

14. The method of claim 11, wherein:
the hybridization step comprises contacting the amplification products with the at least two oligonucleotide probes that are each labeled with a donor fluorescent moiety and a corresponding acceptor moiety; and
the detection step comprises detecting the presence or absence of fluorescence resonance energy transfer (FRET) between the donor fluorescent moiety and the acceptor moiety of the probes, wherein the presence or absence of fluorescence is indicative of the presence or absence of MPXV in the sample.

15. The method of claim 14, wherein the detectable probes are each labeled with a same donor fluorescent moiety.

16. The method of claim 14, wherein the detectable probes are each labeled with a different donor fluorescent moiety.

17. The method of claim 11, wherein the amplification step employs a polymerase enzyme having 5′ to 3′ nuclease activity.

18. The method of claim 11, wherein the sample is a biological sample selected from a skin swab, a lesion swab and plasma.

19. (canceled)

20. A kit for detecting a first target nucleic acid within the F3L gene of MPXV and a second target nucleic acid within the B21R gene of MPXV in a sample, the kit comprising amplification reagents comprising:
(a) a DNA polymerase having 5′ to 3′ nuclease activity;
(b) nucleoside triphosphates;
(c) a first set of oligonucleotide primers and a first oligonucleotide probe or first set of oligonucleotide probes for amplifying and detecting the F3L gene target nucleic acid of MPXV, wherein the first set of oligonucleotide primers for amplification of the F3L gene target nucleic acid comprises a forward primer comprising a nucleic acid sequence selected from the group consisting of SEQ ID NOs: 7-10, or any combination of forward primers each comprising a nucleic acid sequence selected from the group consisting of SEQ ID NOs: 7-10; and a reverse primer comprising a nucleic acid sequence selected from the group consisting of SEQ ID NOS: 11-12, or a combination thereof; and wherein the first probe or first set of probes for detection of an amplification product of the F3L gene target nucleic acid comprises a nucleic acid sequence of SEQ ID NO: 13, or a complement thereof; and
(d) a second set of oligonucleotide primers and a second oligonucleotide probe or second set of oligonucleotide probes for amplifying and detecting the B21R gene target nucleic acid of MPXV, wherein the second set of oligonucleotide primers for amplification of the B21R gene target nucleic acid comprises a forward primer comprising a nucleic acid sequence selected from the group consisting of SEQ ID NOs: 22-25, or any combination of forward primers each comprising a nucleic acid sequence selected from the group consisting of SEQ ID NOs: 22-25; and a reverse primer comprising a nucleic acid sequence selected from the group consisting of SEQ ID NOs: 26-28, or any combination of reverse primers each comprising a nucleic acid sequence selected from the group consisting of SEQ ID NOs: 22-25; and wherein the second probe or second set of probes for detection of an amplification product of the B21R gene target nucleic acid comprises a nucleic acid sequence of SEQ ID NO: 29, or a complement thereof.

21. The kit of claim 20, wherein the first and second oligonucleotide probes or the first set and second set of oligonucleotide probes are labeled with a donor fluorescent moiety and a corresponding acceptor moiety.

22. The kit of claim 21, wherein the first and second oligonucleotide probes are each labeled with a same donor fluorescent moiety or each labeled with a different donor fluorescent moiety.

23-24. (canceled)
